(19) United States
(12) Patent Application Publication
(10) Pub. No.: US 2025/0228949 A1
(43) Pub. Date: Jul. 17, 2025

(54) COLLAGEN MATRICES OF VARYING CHARGE AND CROSS-LINKING DENSITY

(71) Applicant: Integra LifeSciences Corporation, Princeton, NJ (US)

(72) Inventors: Steven L. Bennett, Cheshire, CT (US); Liam Sweeney, Westerly, RI (US); Bryan Vayda, Lawrenceville, NJ (US)

(21) Appl. No.: 19/016,018

(22) Filed: Jan. 10, 2025

Related U.S. Application Data
(60) Provisional application No. 63/620,425, filed on Jan. 12, 2024.
(60) Provisional application No. 63/663,412, filed on Jun. 24, 2024.
(60) Provisional application No. 63/742,542, filed on Jan. 7, 2025.

(51) Int. Cl. A61K 47/42; A61K 9/00; A61K 38/18; A61P 19/08

(52) U.S. Cl. CPC A61K 47/42; A61K 9/0024; A61K 38/1875; A61P 19/08

(57) ABSTRACT

Collagen matrices of varying charge and cross-linking density are disclosed, and methods for preparing such collagen matrices. The cross-linking methods for collagen impart charge and allow for variable cross-linking density within the collagen device. Such variable cross-linking density imparts variable collagen resorption time, and the variable charge allows sequestering and then releasing therapeutics in a controlled manner. The techniques may be used to impact in vitro mineralization via the charged collagen platform by its interaction with rhBMP-2. The techniques can be used to create a positively charged collagen skin substitute that resists infection and supports wound closure.

### CROSS-REFERENCE TO RELATED APPLICATIONS

This application claims priority to U.S. Provisional Patent Application Ser. No. 63/620,425, filed Jan. 12, 2024, and U.S. Provisional Patent Application Ser. No. 63/663,412, filed Jun. 24, 2024, and U.S. Provisional Patent Application Ser. No. 63/742,542, filed Jan. 7, 2025, the entire disclosures of which are incorporated by reference herein.

### BACKGROUND

The present invention relates to medical devices for drug delivery and tissue regeneration. The invention also relates to methods for making or using these devices.

Biomaterials, such as collagen, have been used in tissue regeneration and drug delivery. Collagen is a major protein component of bone, cartilage, skin, and connective tissue in animals. Examples of collagen devices and their uses in tissue regeneration have been described in U.S. Pat. Nos. 5,997,895 and 8,846,060, which are incorporated herein by reference. A sheet of collagen matrix may be produced from a process comprising the steps of preparing a dispersion of collagen, lyophilizing the collagen dispersion to dryness, and then optionally cross-linking the lyophilized collagen, for example, by a glutaraldehyde solution or formaldehyde vapor. Collagen materials are also known to be useful as carriers for drug and protein delivery.

While collagen matrices are known to be able to carry therapeutics agents and release them to the body in a physiological environment, the binding of such therapeutic agents to collagen matrices is not optimal and thus, the release of such therapeutic agents from the collagen matrices may be excessive or off-target. Therefore, it is desirable to develop collagen devices that provide enhanced control of the binding and subsequent release of therapeutics agents. It is also desirable that the degradability of the collagen devices can be modulated.

### SUMMARY

The present disclosure provides a cross-linking method for collagen that can impart charge to the collagen. The cross linkers are water soluble solids that can be dissolved into collagen suspension prior to lyophilization. This allows for varying cross-linking density within a continuous lyophilized collagen foam. Variable cross-linking density imparts variable resorption time.

The cross linkers can also carry a negative or positive charge which can vary charge density of cross-linked collagen foam. The charge may sequester proteins and drugs in a specific manner along the surface of a collagen device. The charge can hold onto therapeutics and then release them over time. The varying charge in collagen devices can also be used to direct cell growth in a specific direction.

The collagen technology platform of the present invention allows for varying the charge and degradation profile of collagen using water soluble cross-linkers and provides a convenient way of cross-linking collagen that can be used to deliver charged substances for tissue regeneration or treatment.

In an aspect of the present disclosure, there is provided a medical device comparing a cross-linked collagen matrix having a positive or negative charge. The collagen matrix is crossed-linked by a positively or negatively charged cross-linker. Examples of cross-linkers include dextran aldehyde, DEAE-dextran aldehyde (positively charged), and CM-dextran aldehyde (negatively charged), or combinations thereof.

In another aspect of the present invention, there is provided a method of controlling release of a physiologically or pharmaceutically active ingredient in a subject, comprising administering to the subject a drug delivery device comparing a cross-linked collagen matrix having a positive or negative charge, and a physiologically or pharmaceutically active ingredient having an opposite charge.

In a further aspect of the present invention, there is provided a method for preparing a drug delivery device comparing a cross-linked collagen matrix having a positive or negative charge, comprising providing a collagen dispersion; adding to the collagen dispersion a positively or negatively charged cross-linker to generate a mixture; lyophilizing the mixture, cross-linking the collagen; and optionally heating the mixture.

In yet another aspect of the present invention, there is provided a medical device comparing a first cross-linked collagen matrix portion having a positive or negative charge; and a second cross-linked collagen matrix portion having a positive or negative charge which is different from the charge of the first cross-linked collagen matrix portion in the type and/or level of charge.

In a further aspect of the present invention, there is provided a medical device comparing a first cross-linked collagen matrix portion having a first charge, first charge density, and first cross-linking density; and a second cross-linked collagen matrix portion having a second charge, second charge density, and second cross-linking density; wherein the first cross-linked collagen matrix portion and the second cross-linked collagen matrix portion differ in the type of charge, charge density and/or cross-linking density.

In a further aspect of the present invention, there is provided a method for treating a wound in a patient. The method includes applying a positively charged cross-linked collagen scaffold to the wound, wherein the collagen scaffold facilitates wound healing while providing antimicrobial resistance. The positively charged cross-linked collagen scaffold is generated by applying diethyl aminoethyl-dextran aldehyde (DEAE-DA (+)) to a purified Type I collagen-based matrix. The method creates a skin substitute that has antimicrobial activity toward Gram-positive bacteria.

These and other features and advantages of the invention or certain embodiments of the invention will become more apparent from the following disclosure and description of exemplary embodiments.

### BRIEF DESCRIPTION OF THE DRAWINGS

Example embodiments of the present invention are described herein with reference to the following figures:

FIG. 1 is a graph showing enzyme degradation time as a function of cross-linking density.

FIG. 2 shows varying charge in a single piece of collagen foam.

FIG. 3 shows varying charge ratios at constant weight %.

FIG. 4 is a graph showing release of negatively charged yellow dye from collagen foams crosslinked at various charge ratios.

FIG. 5 shows a collagen foam where the outer circle contains a higher concentration of neutral dextran aldehyde cross-linker (denoted as 100 (N)) and thus a higher cross-linking density and the inner circle contains a lower concentration of neutral dextran aldehyde (denoted as 12.5 (N)) and thus a lower cross-linking density. FIG. 5 illustrates varied enzymatic degradation and charge of collagen based on cross-linking density within one continuous piece of collagen.

FIG. 6 illustrates a collagen foam scheme with four collagen foams of varying charge provided. The collagen foams are soaked in a solution of blue or violet dyes with a positive charge and yellow dyes with a negative charge. The negatively charged collagen sequesters the positively charged violet or blue dye while the positively charged collagen sequesters the negatively charged yellow dye.

FIG. 7 illustrates a collagen foam where the outer circle contains positively charged DEAE-dextran aldehyde cross-linker and the inner circle contains neutral dextran-aldehyde cross-linker. FIG. 7 illustrates varied charge within one continuous piece of collagen and preferential sequestration of negatively charged BSA to the positively charged region.

FIG. 8 is a plot of neutral cross-linked collagen (denoted as neutral), negatively charged cross-linked collagen (denoted as CM), and positively charged cross-linked collagen (denoted as DEAE) and the amount of epidermal growth factor (EGF) that each foam binds from a stock solution.

FIG. 9 is a plot of neutral cross-linked collagen (denoted as neutral), negatively charged cross-linked collagen (denoted as CM), and positively charged cross-linked collagen (denoted as DEAE) and the amount of platelet-derived growth factor BB (PDGF-bb) that each foam binds from a stock solution.

FIG. 10 is a plot of neutral cross-linked collagen (denoted as N), positively charged cross-linked collagen (denoted as DEAE), and a control absorbable collagen sponge cross-linked with formaldehyde (denoted as ACS) and the amount of fluorescently tagged bovine serum albumin (BSA) that each foam binds from a stock solution.

FIG. 11 is a plot of the release of fluorescently tagged BSA across 7 days from neutral dextran cross-linked collagen (denoted as N), positively charged dextran cross-linked collagen (denoted as DEAE), and a control absorbable collagen sponge cross-linked with formaldehyde (denoted as ACS).

FIG. 12 is a plot of the amount of fluorescently tagged BSA on DEAE (+), ACS, and N foam from FIGS. 10 and 11 after 7 days of release in PBS at 37° C.

FIG. 13 is an example of degradation control over time for a collagen foam scheme.

FIG. 14 is an illustration of aldehyde substitution results.

FIG. 15 is a graph illustrating the effect of concentration of cross-linker and oxidation level on thermal transition temperature, which is representative of collagen cross-linking density. The graph illustrates that the transition temperature increases with increased concentration of cross-linker.

FIG. 16 is a graph illustrating a release of BMP-2 from a negatively charged collagen foam.

FIG. 17 illustrates a process for bone remodeling via rhB MP-2.

FIG. 18 are example images of using elemental mapping (EDS-SEM) to characterize the osteogeneic potential of human mesenchymal stem cells (hMSCs) with BMP-2 via imaging of calcium mineralization.

FIG. 19 is a plot of Alkaline Phosphatase (ALP) activity of human mesenchymal stem cells (MSCs) seeded on collagen foams of varying charge formulations with varying doses of BMP-2 after 7 days in culture.

FIG. 20A and FIG. 20B are plots of Alkaline Phosphatase (ALP) activity of human pre-osteoblasts cells seeded on collagen foams of varying charge formulations dosed with 100 ng of BMP-2 after 5 days in culture (FIG. 20A) and 7 days in culture (FIG. 20B).

FIG. 21 illustrates off-target effects resulting from high BMP-2, specifically adipogenesis.

FIG. 22 and FIG. 23 are plots illustrating the effect of negatively charged collagen on lipid deposition, quantified by leptin secretion.

FIG. 24 illustrates perilipin staining to target maturing adipocytes.

FIG. 25 is a graph illustrating log reduction results for bacteria on charged collagen.

### DETAILED DESCRIPTION

Collagen occurs in several types, having differing physical properties. The most abundant types are Types I, II, and III. In an exemplary embodiment of the present invention, Type I collagen is used for preparing biodegradable porous polymeric matrices. Collagen derived from any source is suitable for use in the compositions of the present invention, including insoluble collagen, collagen soluble in acid, collagen soluble in neutral or basic aqueous solutions, as well as those collagens that are commercially available. Typical animal sources for collagen include but are not limited to recombinant collagen, fibrillar collagen from bovine, porcine, ovine, caprine and avian sources as well as soluble collagen from sources such as cattle bones and rat tail tendon.

The terms “matrix” and “scaffold” as used herein refer to a construct of natural or synthetic polymeric materials which can be used in vivo and in vitro as structural supports for cells and tissues, frameworks for tissue formation and regeneration, or surfaces for cell contact. Collagen matrices may be in any solid form or shape, for example, in the form of foam, membrane, sheet, film, or the like.

The phrase “negatively charged” or “negatively-charged” as used herein means that the molecule or polymer has a negative charge at pH 7.0. The phrase “positively charged” or “positively-charged” as used herein means that the molecule or polymer has a positive charge at pH 7.0.

This invention provides alternative cross-linking methods for collagen that can also impart charge. The cross-linkers are water soluble solids that can be dissolved into collagen suspension or dispersion prior to lyophilization. This allows for variable cross-linking density within a single continuous lyophilized collagen foam. Variable cross-linking density imparts variable resorption time allowing for new elements of the scaffold to resolve over time such as holes or channels. The cross-linkers employed may have a more favorable toxicity profile as compared to formaldehyde or glutaraldehyde.

The charged collagen platform of the present invention is based on three different cross-linkers of collagen: dextran aldehyde, DEAE-dextran aldehyde (positively charged), and CM-dextran aldehyde (negatively charged), as shown in structures (a)-(c) below. The charged collagen foams are manufactured using collagen suspension in combination with three different cross-linking molecules: dextran aldehyde, diethylaminomethyl (DEAE) dextran aldehyde, and carboxymethyl (CM) dextran aldehyde.

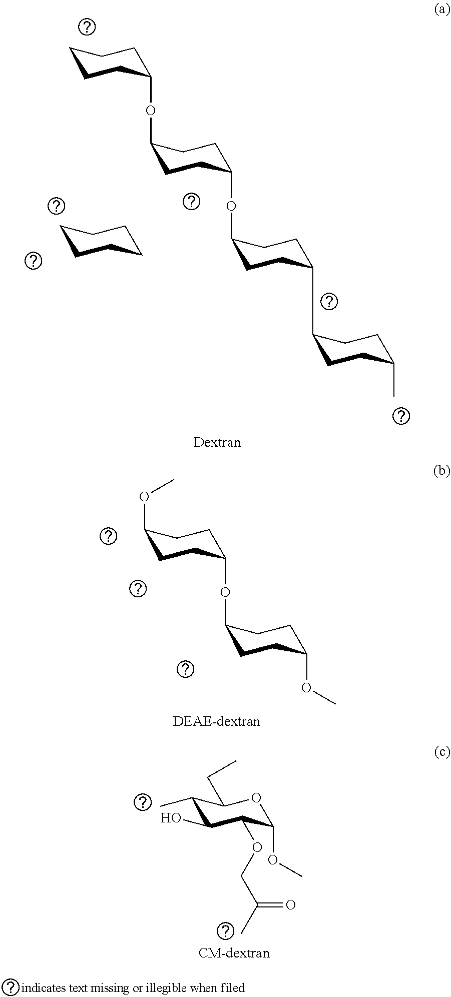

The same principle may be used to synthesize each cross-linker. Sodium periodate is used to oxidize each polysaccharide and substitute aldehyde groups. Oxidation of dextran, DEAE-dextran, and CM-dextran occurs by the addition of sodium periodate (NaIO₄) and results in the formation of aldehyde groups which bind to the amine groups of the collagen peptide to form cross-links. An overview of this oxidation reaction can be found in formula (IV) below.

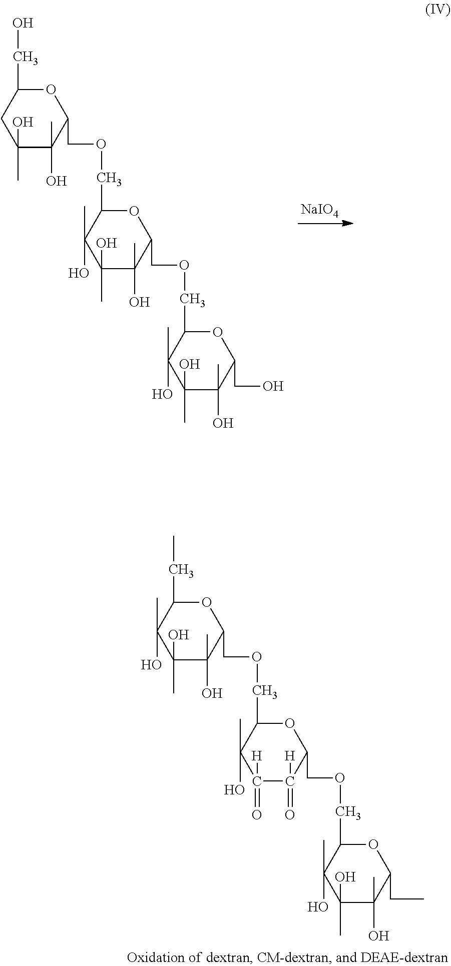

In addition to periodate, other suitable oxidizing agents may also be used.

After oxidation of the dextran molecule is completed, the solution is dialyzed and lyophilized to yield a powder which can be resuspended in water prior to adding to collagen dispersions.

Each lyophilized cross-linker is then dissolved in water and mixed with collagen suspension. The collagen dispersions containing the cross-linker are lyophilized and may be further treated with a de-hydrothermal treatment (DHT) for an additional period of time to create the charged collagen foams.

Synthesis of charged collagen foams is described in more detail below. For producing neutral collagen foams, 1.5 g of oxidized dextran cross-linker is dissolved in 10 mL of deionized water, added to 200 mL of collagen suspension, and shaken. The cross-linked suspension is then poured in lyophilization trays and lyophilized. For producing positively charged collagen foams, 1.5 g of oxidized DEAE-dextran cross-linker is dissolved in 10 mL of deionized water, added to 200 mL of collagen suspension, and shaken. The cross-linked suspension is then poured in lyophilization trays and lyophilized. For producing negatively charged collagen foams, 1.5 g of oxidized CM-dextran cross-linker is dissolved in 10 mL of deionized water, and added to 200 mL of collagen suspension, and shaken. The cross-linked suspension is then poured in lyophilization trays and lyophilized. Once lyophilized, the foams or films are fully cured by heating under vacuum to remove water and drive the reaction to completion.

The cross-linking reaction is a Schiff-Base reaction that occurs between the substituted aldehydes and primary amines on collagen. This reaction produces water as an output, causing the lyophilization and DHT treatment to drive the cross-linking reaction to completion (Formula I). The final product is a lyophilized collagen foam. By cross-linking the charged dextran aldehydes (DEAE-dextran aldehyde or CM-dextran aldehyde), it is possible to create collagen foams of varying charge due to the charged groups on each polysaccharide (carboxymethyl or diethylaminoethyl). DEAE-dextran has been shown to have three function groups with pKa's of 5.5, 9.2, and 14 while CM-Dextran has been shown to have a function group at the carboxymethyl with a pKa of 8.2.

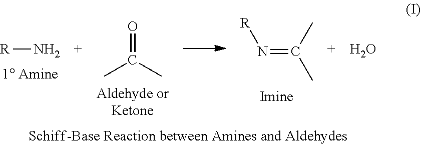

The oxidized DEAE-dextran cross-linked collagen foams have a net positive charge, and the oxidized CM dextran cross-linked collagen foams have a net negative charge. The oxidized dextran cross-linked collagen foams have a closer to neutral net charge relative to the other formulations.

In theory, the charge and cross-linking density of these foams may be varied independently through manipulation of the cross-linking reaction. By adjusting the degree to which each cross-linker is oxidized, the amount of aldehydes per weight of cross-linker can be adjusted, thus increasing (higher oxidation levels) or decreasing (lower oxidation levels) the number of cross-linking sites per repeating unit of the cross-linker. The charge may be varied independently, by adding more or less mass of cross-linker to collagen dispersion prior to lyophilization.

Cross-linking density of the neutral, positively charged, and negatively charged collagen foams can be varied by diluting the initial cross-linked suspensions. 30 g samples of 100%, 75%, 50%, 25%, and 12.5% cross-linking density were made by diluting the initial cross-linked suspensions with the collagen suspension. A control group was also made that consisted of 30 g samples of the collagen suspension alone. Each 30 g sample was mixed and poured into an aluminum weight boat and lyophilized. Table 1 below outlines the quantities for the dilutions used to adjust the cross-linking density. Quantities of cross-linked collagen suspensions and collagen suspensions used to vary cross-linking density of the charged collagen foams are indicated below.

TABLE 1

| Cross-Linking Density | Cross-Linked Suspension | | Collagen Suspension | |
|---|---|---|---|---|
| 100% | 30 | g | 0 | g |
| 75% | 22.5 | g | 7.5 | g |
| 50% | 15 | g | 15 | g |
| 25% | 7.5 | g | 22.5 | g |
| 12.5% | 3.75 | | 26.25 | g |
| 0% | 0 | g | 30 | g |

The net charge of collagen foams is adjusted by mixing the oxidized DEAE-dextran cross-linked suspension and the oxidized CM-dextran cross-linked suspension at different ratios. 30 g samples were made at the ratios and quantities described in Table 2 below. Each sample was shaken, poured into an aluminum weigh boat, and lyophilized. Quantities of oxidized DEAE-dextran cross-linked suspension and oxidized CM-dextran cross-linked suspensions used to vary the net charge of the collagen foams are indicated below.

TABLE 2

| Oxidized DEAE-dextran Cross- | | | Oxidized CM-dextran Cross- | | |
|---|---|---|---|---|---|
| Linked Suspension | | | Linked Suspension | | |
| Percentage | Weight | | Percentage | Weight | |
| 100% | 30 | g | 0% | 0 | g |
| 90% | 27 | g | 10% | 3 | g |
| 80% | 24 | g | 20% | 6 | g |
| 70% | 21 | g | 30% | 9 | g |
| 60% | 18 | g | 40% | 12 | g |
| 50% | 15 | g | 50% | 15 | g |
| 40% | 12 | g | 60% | 18 | g |
| 30% | 9 | g | 70% | 21 | g |
| 20% | 6 | g | 80% | 24 | g |
| 10% | 3 | g | 90% | 27 | g |
| 0% | 0 | g | 100% | 30 | g |

The charge may be localized in collagen matrices. The ability to create regions of varying charge has been examined. This examination is performed by placing a solid mold against a lyophilization tray and pouring 25 g of collagen suspension containing oxidized DEAE-dextran cross-linker around the mold and freezing the suspension. Then, the mold is removed and 5 g of collagen suspension containing oxidized CM-dextran cross-linker is poured into the middle of the tray, replacing the mold. This process creates a small inner circle of oxidized CM-dextran collagen suspension surrounded by oxidized DEAE-dextran collagen suspension that is lyophilized to create a collagen foam with an inner circle of negatively charged, oxidized CM-dextran cross-linked collagen surrounded by positively charged, oxidized DEAE-dextran cross-linked collagen.

Methods for increasing charge have been examined. To add additional negative charge to a collagen foam, carboxymethylcellulose (CMC) sodium salt may be added to a cross-linker collagen suspension. This is done by adding 1.5 g of CMC sodium salt to 10 mL of DI water and stirring until in solution. The CMC sodium salt solution is then added to the 200 mL of suspension. The suspension is shaken and lyophilized. This method was used to add additional charge to oxidized dextran cross-linked suspension and oxidized CM-dextran cross-linked suspension. Other anionically charged molecules, such as bovine serum albumin (BSA), unoxidized CM-dextran, or other anionically charged polymers, may also be included to add additional negative charge to a collagen foam.

To add additional positive charge to a collagen foam, cationic molecules, such as polyethylenimine, more unoxidized DEAE-dextran, polyhexanide (polyhexamethylene biguanide, PHMB), or other cationically charged polymers, may be included to add additional positive charge to oxidized dextran cross-linked suspension and oxidized DEAE-dextran cross-linked suspension. For clarity, the term “cross-linked suspension” as used herein refers to a suspension to which a cross-linker is added or a suspension that contains a cross-linker. The suspension is not necessarily actually cross-linked.

The cross-linkers can also carry a negative or positive charge, which in a similar manner to cross-linking density can vary charge density. This charge may sequester proteins and factors in a specific manner along the surface of a collagen foam or film. This varied charge density can be used to encourage cell growth in a specific direction as in a nerve conduit. This charge can also hold onto and then release therapeutics over time. In addition, this charge can be used in a repulsive fashion to move factors or small molecules in a specific direction for unidirectional drug delivery.

Cross-linking in a charged polymer through chemical modification allows control of net charge and cross-linking density, and thus persistence, independently.

As previously described and illustrated in the examples below, neutral, positive, and negatively charged crosslinkers can be prepared via oxidation of dextran with sodium periodate to form a dialdehyde along the polysaccharide backbone. For example, positively charged cross linkers can be prepared from DEAE (diethylaminoethyl) dextran, and negatively charged cross linkers can be prepared from CM (carboxymethyl) dextran. Additional cross linkers that may be used in the present invention include other polysaccharides and sugars. For example, positively charged cross linkers can be prepared from cationic or positively charged polysaccharides, such as chitosan. Negatively charged cross linkers can additionally be prepared from anionic or negatively charged polysaccharides, such as dextran sulfate, hyaluronic acid, heparin, xanthan-gum, pectin, homogalacturonan, type I rhamnogalacturonan, arabinan-rich Pectic polysaccharides, galactan-rich pectic polysaccharides, type I arabinogalactans, type II arabinogalactans, rhamnogalacturonan (RG-II)-rich pectic polysaccharides, arabinogalactan protein, xylan, arabioxylans, 2-carrageenan, 1-carrageenan, K-carrageenan, porphyrin, agarose, alginate, fucoidan, ulvan, mannoprotein, etc. Neutral cross-linkers can additionally be prepared from neutral polysaccharides such as galactomannan, glucomannan, starch, xyloglucans, laminarans, curdlan, etc.

These cross linkers can be used to control the cross-linking density of collagen by controlling the degree of oxidation of the cross linker itself as well as the weight percentage (wt %) of the cross linker dissolved into a collagen suspension.

Because the degree of oxidation can be controlled in this invention, charge density is decoupled from cross-linking density.

Although the invention is described herein with examples of collagen materials, the invention can also be applied to other biocompatible materials, including synthetic or natural polymers, such as water-soluble polyamine materials or extracellular matrices, other macromolecules. Examples include glycosaminoglycans such as hyaluronans, chondroitin sulfate, dermatan sulfate, heparin sulfate, keratin sulfate, proteoglycans such as aggrecan, decorin, syndecans, as well as elastin, fibronectin, laminin-1, and laminin-2.

One or more drugs may be released from the charged collagen of the present disclosure at varying times. Examples of suitable drugs which may be delivered by a charged collagen matrix of the present disclosure include, but are not limited to, antimicrobial agents, protein and peptide preparations, antipyretic, antiphlogistic and analgesic agents, anti-inflammatory agents, vasodilators, antihypertensive and antiarrhythmic agents, hypotensive agents, antitussive agents, antineoplastic agents, local anesthetics, hormone preparations, antiasthmatic and antiallergic agents, antihistaminics, anticoagulants, antispasmodics, cerebral circulation and metabolism improvers, antidepressant and antianxiety agents, vitamin D preparations, hypoglycemic agents, antiulcer agents, hypnotics, antibiotics, antifungal agents, sedative agents, bronchodilator agents, antiviral agents, dysuric agents, glycosaminoglycans, carbohydrates, nucleic acids, inorganic and organic biologically active compounds, combinations thereof, and the like. Specific biologically active agents include, but are not limited to, enzymes, angiogenic agents, anti-angiogenic agents, growth factors, antibodies, neurotransmitters, psychoactive drugs, anticancer drugs, antimicrobial agents including antibiotics such as rifampin, chemotherapeutic drugs, drugs affecting reproductive organs, genes, oligonucleotides, combinations thereof, and the like.

The charged collagen and methods of the present invention may be used in the following additional applications, for example, using these formulations as inks for 3D printing, unidirectional drug delivery using laminated collagen of varying charge to push therapeutics towards target tissue, creating a charge gradient along a tube of collagen by varying the feed ratio of +/−/neutral, creating pathways for neovascularization in a collagen scaffold via controlled crosslinking, lining those newly exposed pathways with a protein held in place by charge, or natural sequestration of factors based on charge in vivo. Additional examples of applications include using the charged collagen scaffolds of the present invention as an antimicrobial collagen scaffold, using these formulations to enhance regeneration of bone by preferential sequestration and delivery of BMP-2, using these formulations to enhance treatment of chronic wounds by preferential sequestration and delivery of PDGF-bb, or using the collagen graft materials of the present invention for additional tissue regeneration applications.

The following examples are provided for illustrative purposes only and are in no way intended to limit the scope of the present invention.

### EXAMPLES

### Example 1. Preparation of Collagen Suspension

A collagen dispersion is prepared according to methods described in, for example, U.S. Pat. Nos. 5,997,895, 8,846,060, and 10,806,833, which are all incorporated herein by reference. A native source of Type I collagen, such as skin, tendons, ligaments, or bone, is first mechanically or hand cleaned of fat, fascia, and other extraneous matter and washed. The cleaned and washed collagen containing material is then comminuted, generally by slicing or grinding. The material is then subjected to an enzyme treatment while under intermittent stirring with a proteolytic enzyme, such as ficin, pepsin, and the like, so as to remove non-collagenous impurities that may cause antigenic activity and to swell the collagen by removing elastin. The amount of enzyme added to the collagen material and the conditions under which enzyme digestion takes place is dependent upon the particular enzyme being used. Generally, when using ficin, which is most commonly used, the pH is adjusted to about 6.0 to 6.3, and the collagen material is digested for about 1 to 2 hours at a temperature of about 36.5° C. to 37.5° C. with one part ficin for every 150 parts of collagen material. After the requisite amount of time, the enzyme is inactivated by appropriate means well known in the art, such as by the addition of a solution of an oxidizing agent, such as sodium chlorite when the enzyme is ficin. The enzyme treated collagen containing material is then washed to remove excess enzyme and the non-collagenous protein impurities. Preferably, the washing is carried out with ultrafiltered and deionized water and optionally further washed with dilute aqueous hydrogen peroxide.

The enzyme digested collagen containing material is then further subjected to an alkali treatment at a pH of about 13 to 14, at a temperature of about 25° C. to 30° C., for a period of about 35 to about 48 hours. An exemplary period is about 40 hours. The alkali treatment is carried out in an aqueous solution of 5% sodium hydroxide and 20% sodium sulfate. This alkali treatment removes contaminating glycoproteins and lipids. The solution is then neutralized with a suitable acid, such as aqueous sulfuric acid, and thoroughly washed. The collagen material is then further swollen with a suitable acid solution that does not cause any cross-linking of the collagen. Such acids are well known to those skilled in the art and include acetic acid, hydrochloric acid, lactic acid, and the like. Regardless of which acid is used, the pH of the acid collagen dispersion is in the range of about 2 to 3.

The dispersed collagen mixture is then homogenized by any conventional means, such as a blender or homogenizer, so as to further dissociate the fibers and is then filtered to remove unswollen, non-collagenous material by means well known in the art, such as by passing the dispersion through a 100 mesh stainless steel screen. The resulting filtered collagen dispersion can then be used to prepare the charged collagen matrix of the present invention.

### Example 2. Preparation of Oxidized Dextran Cross-Linkers

The method for creating the charged collagen foams begins with oxidation of dextran, DEAE-dextran, and CM-dextran with sodium periodate.

Step A) Synthesis of Oxidized Dextran: 10 g of dextran from Leuconostoc Mesenteroides is added to 150 mL of deionized water, stirred until in solution, and placed in the freezer at 4° C. for 20 minutes. 1.32 g of sodium periodate is added to the dextran solution while being stirred for 2 hours and maintaining a pH of roughly 5.5. The pH may be adjusted by using 1M sodium hydroxide (NaOH) and 1M hydrochloric acid (HCl). The solution is dialyzed for 3 days using dialysis tubing with a 6,000-8,000 Da molecular weight cut off and lyophilized to create the dry powder form that is used as a cross-linker for the neutral collagen foams.

Step B) Synthesis of Oxidized DEAE-dextran: 10 g of DEAE-dextran hydrochloride is added to 150 mL of deionized water and stirred until in solution and placed in the freezer at 4° C. for 20 minutes. 1.097 g of sodium periodate is added to the solution while being stirred for 2 hours and maintaining a pH of roughly 5.5. The pH may be adjusted by using 1M NaOH and 1M hydrochloric acid. The solution is dialyzed for 3 days using dialysis tubing with a 6,000-8,000 Da molecular weight cut off and lyophilized to create the dry powder form that is used as a cross-linker for the positively charged collagen foams.

Step C) Synthesis of Oxidized CM-dextran: 10 g of CM-dextran sodium salt is added to 150 mL of deionized water and stirred until in solution and placed in the freezer at 4° C. for 20 minutes. 1.215 g of sodium periodate is added to the solution while being stirred for 2 hours and maintaining a pH of roughly 5.5. The pH may be adjusted by using 1M NaOH and 1M hydrochloric acid. The solution is dialyzed for 3 days using dialysis tubing with a 6,000-8,000 Da molecular weight cut off and lyophilized to create the dry powder form that is used as a cross-linker for the negatively charged collagen foams.

The weight of sodium periodate may be adjusted to change the degree to which each polysaccharide is oxidized. For the purposes of this platform, the 5%, 10%, and 15% oxidation levels of dextran have been analyzed. The oxidized dextran is dialyzed in 6-8 kDa molecular weight cutoff (MWCO) dialysis tubing in DI water for 3 days, lyophilized, and stored dry for addition to collagen dispersion.

### Example 3. Preparation of Charged Collagen Devices

The lyophilized dextran aldehyde, from each oxidation level, is reconstituted in 10 ml of water and then added to a collagen dispersion. The cross-linker from each oxidation level is added to collagen at varying concentrations: 15 mg/ml, 11.25 mg/ml, 7.5 mg/ml, 3.75 mg/ml, and 1.875 mg/ml. Each sample is then lyophilized for 22 hours and then treated with a DHT cycle for 24 hours. The DEAE-dextran aldehyde cross-linked collagen and dextran aldehyde cross-linked collagen were treated with DHT at 50° C. for 24 hours, while the CM-dextran aldehyde cross-linked collagen was treated with DHT at 100° C. for 24 hours. Following DHT, each sample was packaged in sample bags and stored for analysis.

### Example 4. Cross-Linking Density

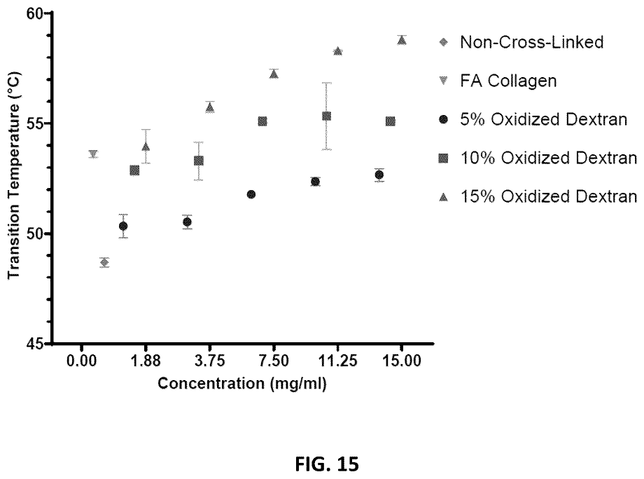

Cross-linking density can be elucidated by the transition temperature with differential scanning calorimetry (DSC) and illustrated in FIG. 15. In most cases the transition temperature increases with increased cross-linking density. FIG. 15 is described in greater detail below.

In the enzymatic degradation assay, the start time of the assay was recorded as the time at which the samples were transferred into the vials of enzyme solution. Over the course of 8 hours, at 37° C., the samples were observed continuously. The time at which particles begin to be observed in the solution was recorded as the fractionation time of each sample. The time at which each sample was no longer visible was recorded as the degradation time.

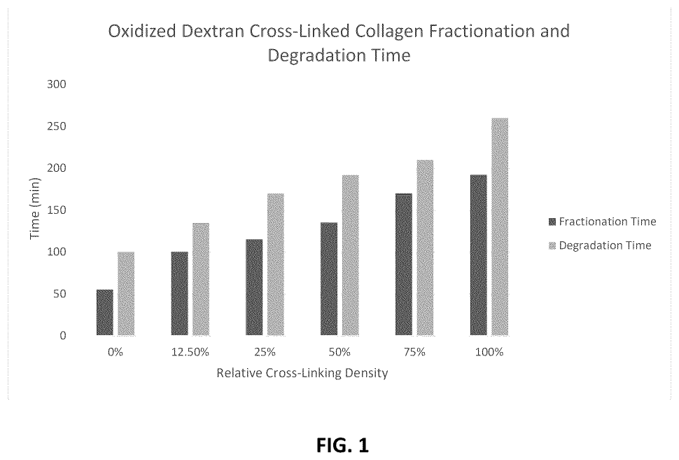

FIG. 1 is a graph showing enzyme degradation time as a function of cross-linking density. In this case, cross-linking density is relative and determined by the weight of dextran aldehyde dissolved in the suspension. CM dextran aldehyde and DEAE dextran aldehyde foams show similar trends.

### Example 5. Collagen Device Having Multiple Cross-Linking Densities

The cross-linking density can be varied within a single piece of lyophilized collagen by freezing a suspension with 100% relative cross-linking density in a mold that has gaps and then filling those gaps with a suspension with 25% relative cross-linking density. The material in the gaps with lighter cross linking will degrade faster in-vivo theoretically. Varying rates of degradation have been demonstrated on bench with collagenase.

### Example 6. Varying Charge Density

The charge density can be controlled by the type and wt % of the cross linker added to the collagen suspension. The charge and cross-linking density can be varied independently by controlling the oxidation level of the polysaccharide. For example, a 1% solution of 10% oxidized DEAE dextran will be cross linked at the same density as a 2% solution of a 5% oxidized DEAE but will have ½ the charge.

### Example 7. Collagen Device Having Multiple Charge Densities

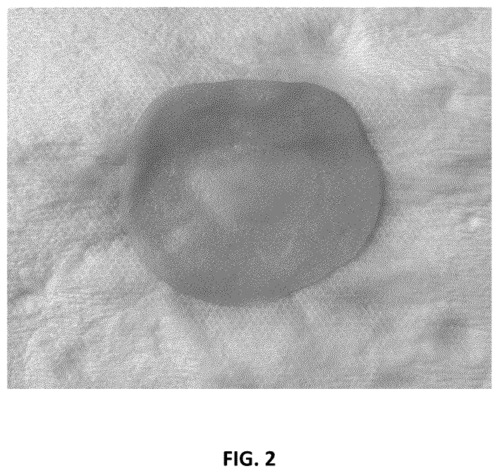

Similar to cross-linking density, charge density can be isolated to varying areas of a single continuous collagen foam by freezing independently and then lyophilizing as a whole. A frozen doughnut shape was formed by pouring DEAE/collagen suspension into a round weigh boat with a solid mold and then freezing. Once frozen the doughnut hole is filled with CM/collagen suspension and the whole frozen assembly is lyophilized and cured. The assembly handles as if one piece but when dipped in a solution of negatively charged yellow dye and positively charged blue dye and rinsed, the foam self-selects for the small molecule based on charge. Divalent cationic and anionic dyes are used to assess the ability of the charged collagen foams to sequester molecules of opposite charge. For this experiment two dyes were used: Methylene Blue Hydrate (net positive charge) and Yellow Acid 17 (net negative charge). FIG. 2 shows yellow at the doughnut circle part and blue at the center part of the collagen foam, indicating varying charge in a single piece of collagen foam.

### Example 8. Use of a Mixture of Cross-Linkers

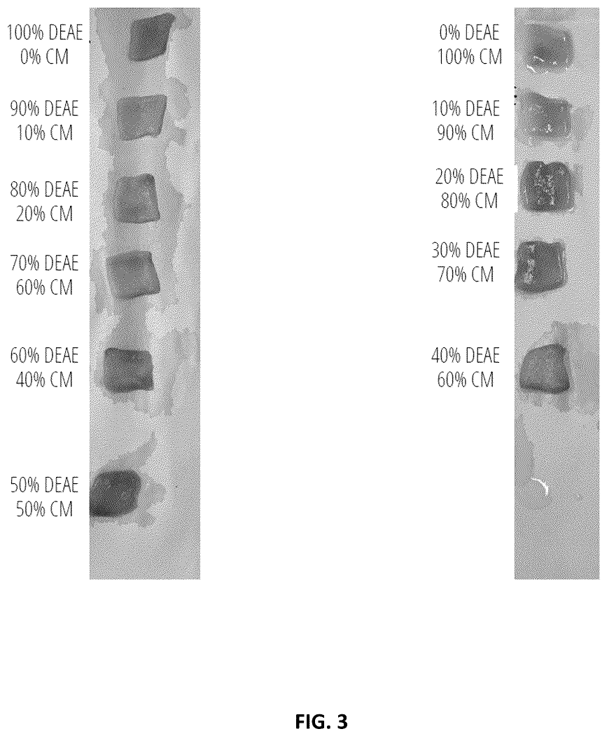

The charged crosslinkers can be mixed in solution at various ratios so they pick up both dyes but in varying degrees which produces a color change from yellow through green to blue as the ratio goes from 0/100 CM/DEAE to 100/0. FIG. 3 shows the varying charge ratios at constant weight %.

### Example 9. Varying Charge and Varying Release Rates

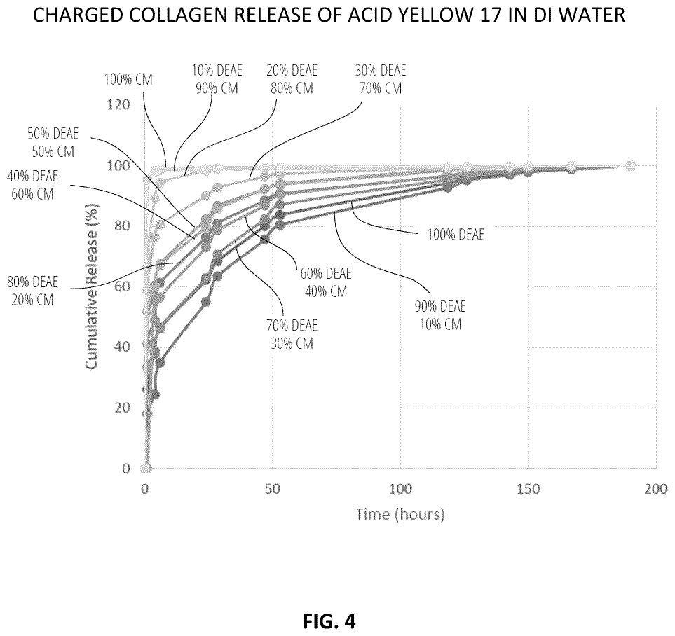

This varying charge will also release the dye at varying rates. If the charge is varied as in the previous example and each foam is independently soaked in a solution of negatively charged yellow dye and tracked by UV/vis over time the plot in FIG. 4 is generated. The foams with a high negative charge release the dye very quickly while the positive charged foams retain the dye due to electrostatic attraction. The data in FIG. 4 was gathered from experiments in DI water at room temperature. These charged collagen foams are not able to retain small molecules with limited point charges, but if the same concept is applied to a polymer or a protein with multiple charge points along the backbone the retention is extended to days and weeks even if run in PBS at body temperature.

### Example 10. Collagen Device Having Varied Cross-Linking Densities

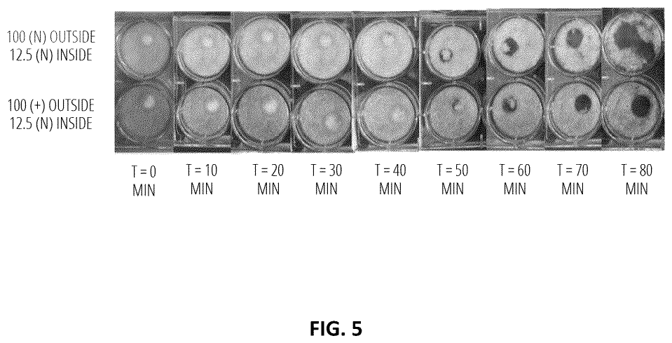

FIG. 5 shows a collagen foam where the outer circle contains a higher concentration of neutral dextran aldehyde cross-linker (denoted as 100 (N)) and thus a higher cross-linking density and the inner circle contains a lower concentration of neutral dextran aldehyde (denoted as 12.5 (N)) and thus a lower cross-linking density. When exposed to a solution containing collagenase, the inner circle is enzymatically degraded first leaving the outer circle behind. Within the same figure but below the top series of images is a collagen foam where the outer circle contains positively charged DEAE-dextran aldehyde cross-linker at a higher concentration (denoted as 100 (+)) and thus a higher cross-linking density and the inner circle contains neutral dextran aldehyde cross-linker at a lower concentration (denoted as 12.5 (N)). When exposed to a solution containing collagenase, the inner circle is enzymatically degraded first leaving the outer, positively charged circle behind. This demonstrates the independent control of charge and degradation of collagen within one continuous collagen foam.

### Example 11. Collagen with Varied Charge and Sequestration of Bovine Serum Albumin

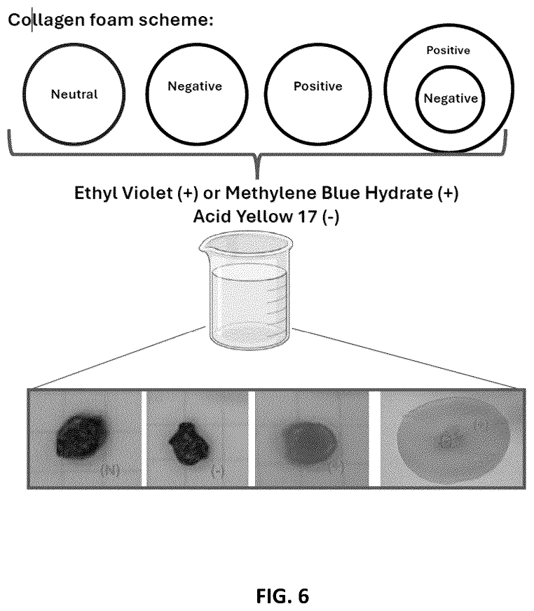

FIG. 6 illustrates a collagen foam scheme where four collagen foams of varying charge are provided. The four collagens are illustrated with a charge of Neutral, Negative, Positive, and Positive with an inner portion that is Negative. Divalent cationic and anionic dyes are used to assess the ability of the charged collagen foams to sequester molecules of opposite charge. For this experiment three dyes were used: Ethyl Violet (net positive charge), Methylene Blue Hydrate (net positive charge), and Yellow Acid 17 (net negative charge).

FIG. 6 illustrates that the neutral collagen foam did not show a significant color change, which indicates that the charge did not sequester a particular dye. The negatively charged collagen foam shows a bright violet color indicating the ability of the negatively collagen to sequester molecules of opposite charge. That is, the negative charge of the collagen sequestered ethyl violet molecules because the ethyl violet molecules have a positive charge. Next, the positively charged collagen shows a yellowish color indicating the ability of the positively charged collagen to sequester molecules of opposite charge, the negatively charged Acid Yellow 17.

The positively charged collagen with an inner portion that is negatively charged shows a yellow outer region with a blue region in the center. The negatively charged inner portion sequestered ethyl violet molecules because the ethyl violet molecules have a positive charge. The outer ring that is positively charged sequestered molecules of opposite charge, the negatively charged Acid Yellow 17.

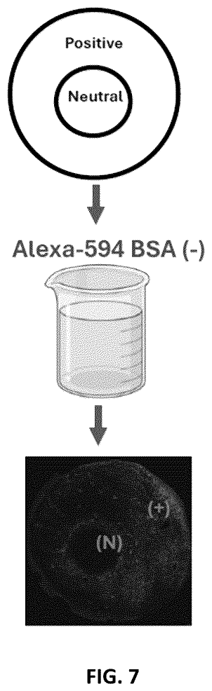

FIG. 7 illustrates a collagen foam scheme in which a collagen foam of varying charge is provided. The collagen is configured with a Positive outer ring with an inner portion that is Neutral. For this experiment the collagen was introduced to Alexa-594 BSA, which is negatively charged. As illustrated the positively charged outer ring sequestered Alexa-594 BSA molecules because the Alexa-594 BSA molecules have a negative charge. The sequestered Alexa-594 BSA molecules are indicated by the purple regions of the image. The inner portion that has a neutral charge did not sequester a significant number of molecules, as illustrated by the black region of the image in the inner portion. The outer positively charged circle has adsorbed more BSA than the inner neutral circle. This result is rational as the positively charged collagen has a higher affinity to the negatively charged BSA. This demonstrates the ability to preferentially sequester molecules of a specific charge to a specific region of a continuous collagen foam.

### Example 12. Preferential Sequestration of Epidermal Growth Factor

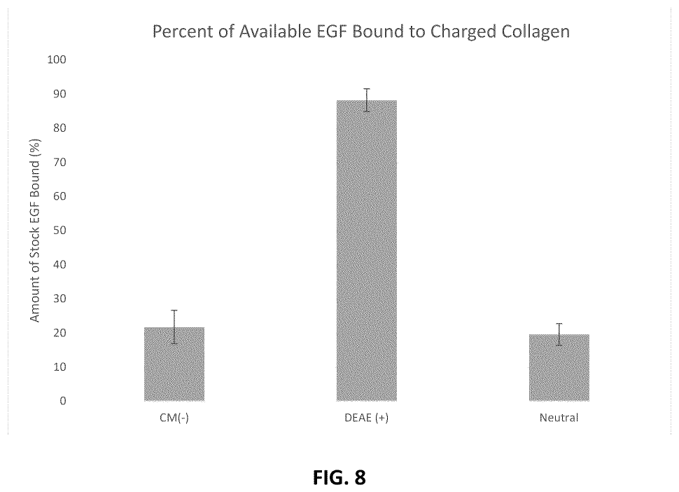

FIG. 8 is a plot of neutral cross-linked collagen (denoted as neutral), negatively charged cross-linked collagen (denoted as CM), and positively charged cross-linked collagen (denoted as DEAE) and the amount of epidermal growth factor (EGF) that each foam binds from a stock solution. EGF has an isoelectric point of 4.6 meaning that it is negatively charged at physiological pH. As demonstrated here, the positively charged collagen adsorbs significantly more EGF than the neutral collagen and the negatively charged collagen.

### Example 13. Preferential Sequestration of Platelet-Derived Growth Factor

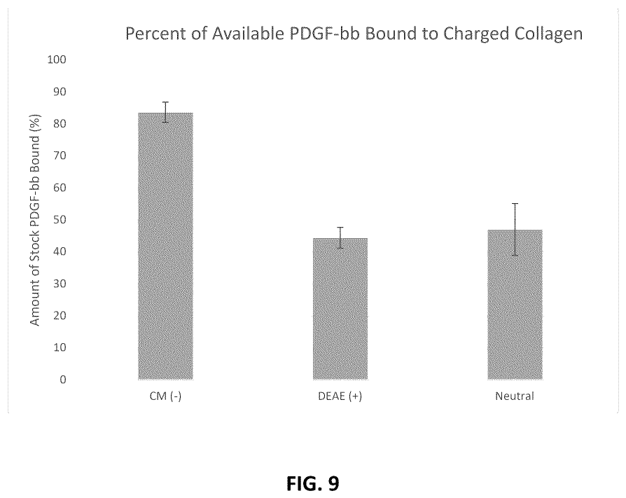

FIG. 9 is a plot of neutral cross-linked collagen (denoted as neutral), negatively charged cross-linked collagen (denoted as CM), and positively charged cross-linked collagen (denoted as DEAE) and the amount of platelet-derived growth factor BB (PDGF-bb) that each foam binds from a stock solution. PDGF-bb has an isoelectric point of 9.8 meaning that it is positively charged at physiological pH. As demonstrated here, the negatively charged collagen adsorbs more PDGF-bb than the neutral collagen and the positively charged collagen.

### Example 14. Preferential Sequestration of BSA

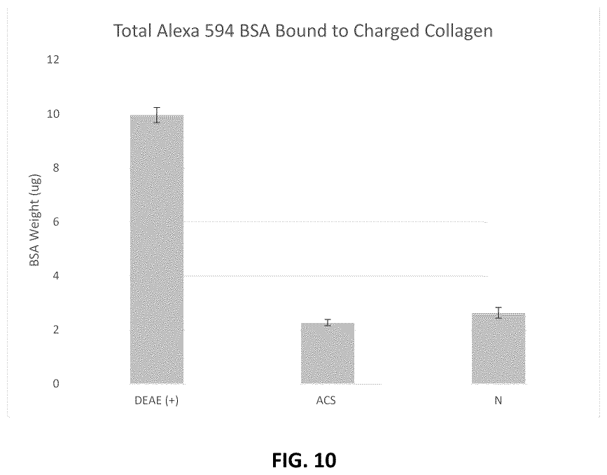

FIG. 10 is a plot of neutral cross-linked collagen (denoted as N), positively charged cross-linked collagen (denoted as DEAE), and a control absorbable collagen sponge cross-linked with formaldehyde (denoted as ACS) and the amount of fluorescently tagged bovine serum albumin (BSA) that each foam binds from a stock solution. BSA has an isoelectric point of 4.5 meaning that the BSA is negatively charged at physiological pH. As demonstrated here, the positively charged collagen adsorbs more BSA than the neutral dextran aldehyde cross-linked collagen and the formaldehyde cross-linked collagen.

### Example 15. Release of Sequestered BSA

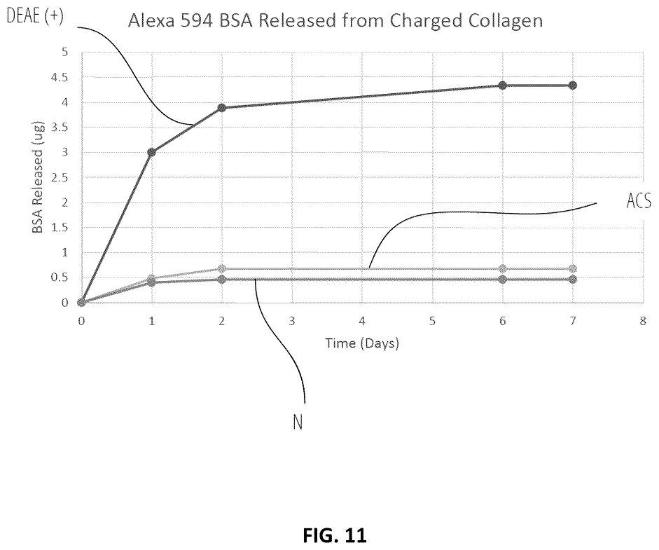

FIG. 11 is a plot of the release of fluorescently tagged BSA across 7 days from neutral dextran cross-linked collagen (denoted as N), positively charged dextran cross-linked collagen (denoted as DEAE), and a control absorbable collagen sponge cross-linked with formaldehyde (denoted as ACS). After soaking in a stock solution of BSA, the samples were placed in PBS at 37° C. The buffer was fully exchanged and the amount of BSA released at each timepoint was determined using a fluorescent calibration curve and a microplate reader.

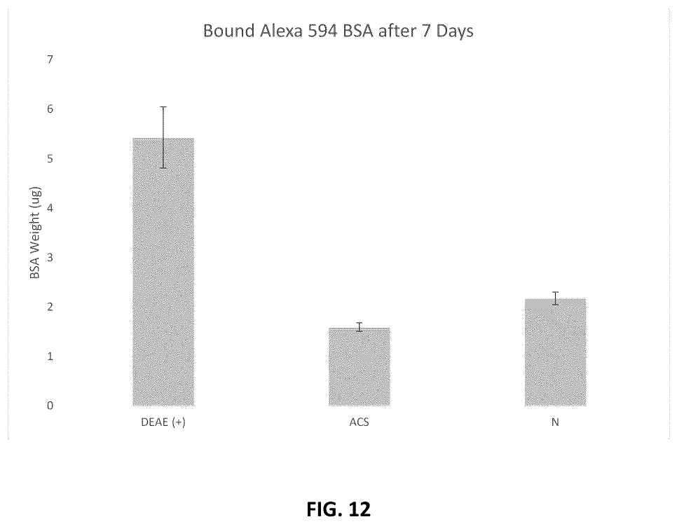

FIG. 12 is a plot of the amount of fluorescently tagged BSA on each foam as described with respect to FIGS. 10 and 11 after 7 days of release in PBS at 37° C. This value was determined by enzymatically degrading each sample and quantification using a fluorescent calibration curve and a microplate reader.

### Example 16. Controlled Delivery of rhBMP-2

Collagen is an attractive scaffold for the delivery of drugs and macromolecules due to collagen's favorable biocompatibility possessing inherent cell attachment sites. However, current engineered collagen systems offer little control over attachment and release of macromolecules or alter the biocompatibility of the collagen through crosslinking. The processes tested in example 16 attempt to develop a simple and robust system for drug delivery through the independent alteration of the charge density and degradation time of collagen. Since macromolecules possess different isoelectric points, the process can exploit the charge density to preferentially sequester and release macromolecules in the resulting collagen scaffold. The tests focus on binding and delivery of positively (rhBMP-2) and negatively charged macromolecules in this system.

After rigorous material characterization of cross-linking to confirm our capacity to independently control net charge and collagen degradation, we investigated the binding and release of rhBMP-2 via UPLC and the potential therapeutic impact by determining the corresponding osteogenic and adipogenic potential to varying rhBMP-2 concentrations with human mesenchymal stromal cells (hMSCs) seeded on charged collagen. Osteogenic capacity was evaluated by Alizarin Red staining (ARS), Alkaline Phosphatase (ALP) activity, and energy dispersive X-ray spectroscopy (EDS). Off-target effects were determined through Oil Red O staining and leptin ELISA for adipogenesis.

After confirming the osteoinductive nature of rhBMP-2 in 2D monolayers with ARS and ALP activity, the tests seeded hMSCs on the engineered collagen scaffolds to determine resulting changes in osteoinduction. The tests indicated that negatively charged collagen promotes a rhBMP-2 sensitive dose response and significantly greater ALP activity than other groups at the higher doses, suggesting that the negative charge sequesters bioactive rhBMP-2 to a greater extent than other collagen foams tested (control ACS, positive, and neutral). The tests confirmed that resulting mineralization on the charged collagen is correlated to calcium deposition through EDS elemental imaging on sample surfaces. Toward an alternative differentiation pathway, the tests recapitulated adipogenic differentiation of hMSCs simply by delivering high rhBMP-2 concentration in osteogenic media. The tests determined that increasing rhBMP-2 concentration from 100 ng/ml by ten-fold increments up to 1 μg/mL triggers an increasing presence of Oil Red O-positive lipid droplets. Further, the leptin ELISA results bore similar rhBMP-2 dose dependency with increased leptin secretion across several engineered collagen scaffolds (ACS, neutral, and negative). However, the negatively charged collagen decreased leptin secretion at higher doses of rhBMP-2.

Altogether, the tests show that a process can impact mineralization via our charged collagen platform by its interaction with rhBMP-2. Moreover, the tests show that high levels of rhBMP-2 can elicit undesirable off-target effects, but that the negatively charged collagen platform may mitigate such off-target effects while improving the osteogenic differentiation of hMSCs. Beyond use with BMP-2, the testing has provided data to support the use of the platform with additional macromolecules of interest (EGF and PDGF-bb). These data have demonstrated that positively charged collagen preferentially sequesters the negatively charged EGF while the negatively charged collagen preferentially sequesters the positively charged PDGF-bb. The testing indicates that this charged collagen platform could be exploited as a tool for several drug delivery applications relying simply on the innate isoelectric properties of a macromolecule of interest.

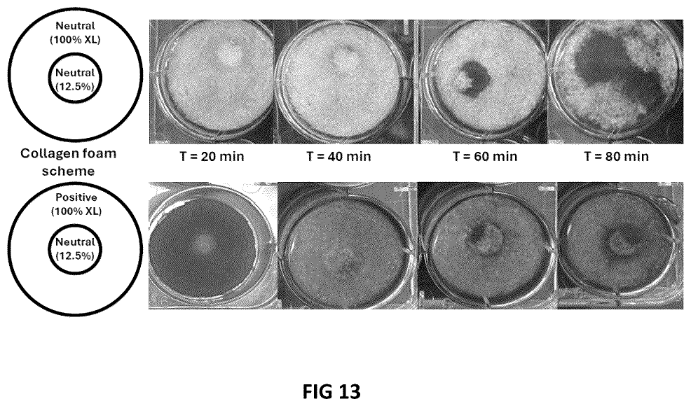

FIG. 13 is an example of degradation control over time for a collagen foam scheme. In the top example, a neutral (100% XL) charge is created on an outer ring with a neutral (12.5% XL) charge in the inner region. The collagen was allowed to degrade over a time period of 80 minutes. The first image is after 20 minutes. The outer ring and inner region are generally still intact with the inner region showing greater cohesion. The second image is after 40 minutes, and the third image is after 60 minutes. The outer ring is showing a greater amount of degradation than the inner region. After 80 minutes, the final image is indicating a degradation of all portions of the collagen foam.

In the lower example, a positive (100% XL) charge is created on an outer ring with a neutral (12.5% XL) charge in the inner region. The collagen was allowed to degrade over a time period of 80 minutes. The first image is after 20 minutes. The outer ring and inner region are generally still intact with the inner region becoming discolored indicating some level of degradation. The second image is after 40 minutes, and the third image is after 60 minutes. The inner region is showing a greater amount of degradation than the outer ring. After 80 minutes, the final image is indicating a slight degradation of all portions of the collagen foam with the inner region experiencing a greater level of degradation.

These examples of FIG. 13 illustrate that the degradation of the collagen foam may be localized by varying the net charge over regions of a singular, continuous collagen material. A localized portion of the foam, such as the center portion may be configured to degrade more quickly or more slowly. The alteration in net charge impacts the binding affinity of model proteins, such as bovine serum albumin (BSA) (−), epidermal growth factor (EGF) (−), and platelet derived growth factor (PDGF-bb) (+).

Aldehyde substitution, thermal transition temperature and enzymatic degradation demonstrate control over the process for collagen cross-linking.

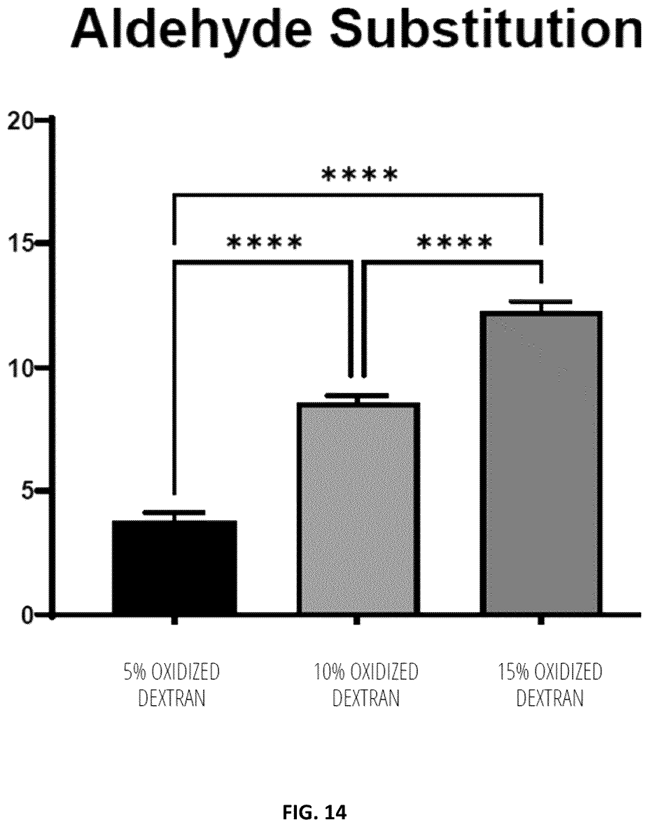

FIG. 14 is an illustration of aldehyde substitution results are illustrated. As described in one example herein, sodium periodate may be used to oxidize each polysaccharide and substitute an aldehyde group. Oxidation of dextran, DEAE-dextran, and CM-dextran occurs by the addition of sodium periodate (NaIO4) and results in the formation of aldehyde groups that bind to the amine groups of the collagen peptide to form cross-links.

In the graph of the FIGS. 14, 5%, 10%, and 15% oxidized dextran was tested. The percent of aldehyde substitution for each sample is displayed as a bar graph. 15% oxidized dextran resulted in approximately 12% aldehyde substitution. 10% oxidized dextran resulted in approximately 8% aldehyde substitution. 5% oxidized dextran resulted in approximately 4% aldehyde substitution.

In FIG. 15, the effect of thermal transition temperature on the process for collagen cross-linking is illustrated. Cross-linking density can be demonstrated by the transition temperature with differential scanning calorimetry (DSC). In most cases, the transition temperature increases with increased cross-linking density.

In the illustration, three samples of 5%, 10%, and 15% oxidized dextran was tested, along with a non-cross-linked sample and an FA collagen sample. Each percentage of dextran was tested at 5 different concentrations.

The transition temperatures generally increased with cross-linking density. For example, for 5% oxidized dextran, the temperature increased from approximately 50.5 degrees C. to approximately 52.5 degrees C. as the concentration increased from approximately 1 mg/ml to approximately 13 mg/ml.

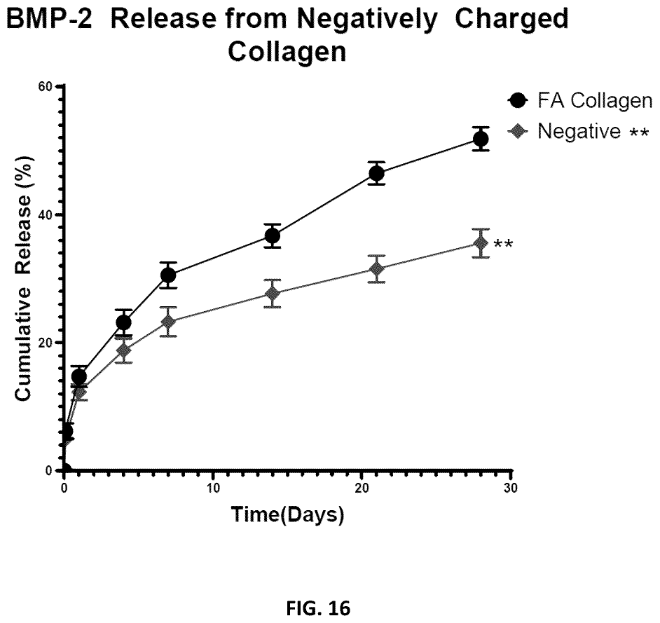

FIG. 16 is a graph illustrating a release of BMP-2 from a negatively charged collagen foam. A release of BMP-2 from an FA collagen is also illustrated for comparison. The foams with the negative charge released the BMP-2 more slowly than the FA collagen foam. For example, after 30 days, the negatively charged collagen foam had a cumulative release percentage of approximately 35% while the FA collagen foam had a cumulative release percentage of approximately 52%.

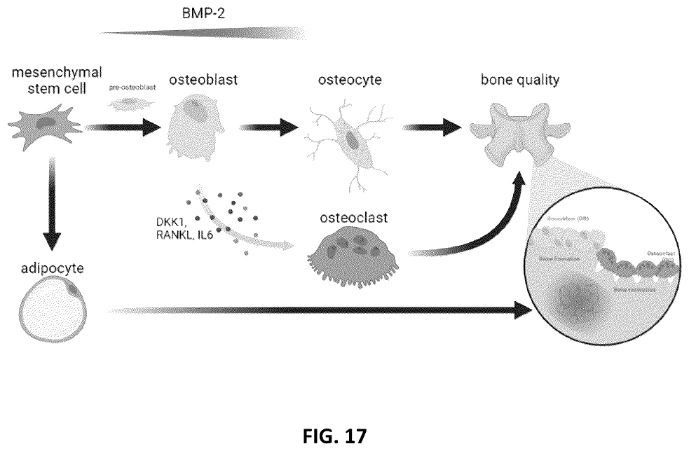

FIG. 17 illustrates a process for bone remodeling via rhBMP-2. The quality of the bone remodeling is impacted by the amount of rhBMP-2.

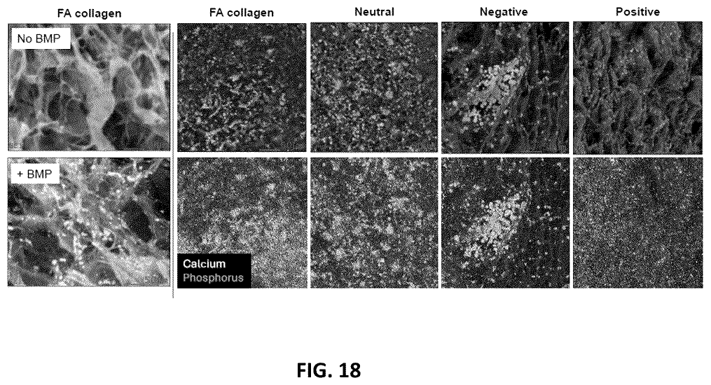

In FIG. 18, displays an example of using elemental mapping (EDS-SEM) to characterize the osteogeneic potential of human mesenchymal stem cells (hMSCs) with BMP-2. As illustrated samples in the presence of BMP-2 and samples not in the presence of BMP-2 are illustrated after 4 weeks. Samples using FA collagen with BMP-2 exhibit dense, white particulates. An overlay with yellow and blue pixels is illustrated with yellow indicating Calcium and blue indicating Phosphorus. The particulates are illustrated for FA collagen, neutral, negative, and positive collagen samples.

### Example 17. Alkaline Phosphatase Activity of Human Mesenchymal Stem Cells

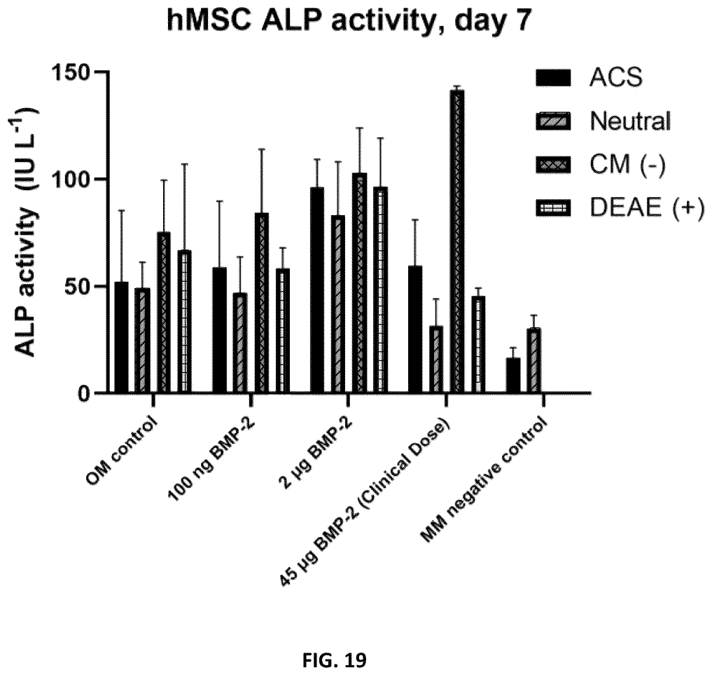

FIG. 19 is a plot of alkaline phosphatase (ALP) activity of human mesenchymal stem cells (MSCs) seeded on collagen foams of varying charge formulations with varying doses of BMP-2 after 7 days in culture. ALP activity is a marker of differentiation of MSCs to osteoblasts and bone formation. ALP activity is enhanced with treatment of BMP-2. The data demonstrates that the negatively charged collagen foam (denoted as CM (−)) maintains higher ALP activity levels than all groups at the highest BMP-2 dose after 7 days indicating that the negatively charge collagen foam enhances the biological effect, osteoinductivity of BMP-2. The data indicates that this result is due to the preferential sequestration and release of the positively charged BMP-2 from the negatively charged collagen.

### Example 18. Alkaline Phosphatase Activity of Human Pre-Osteoblasts Cells

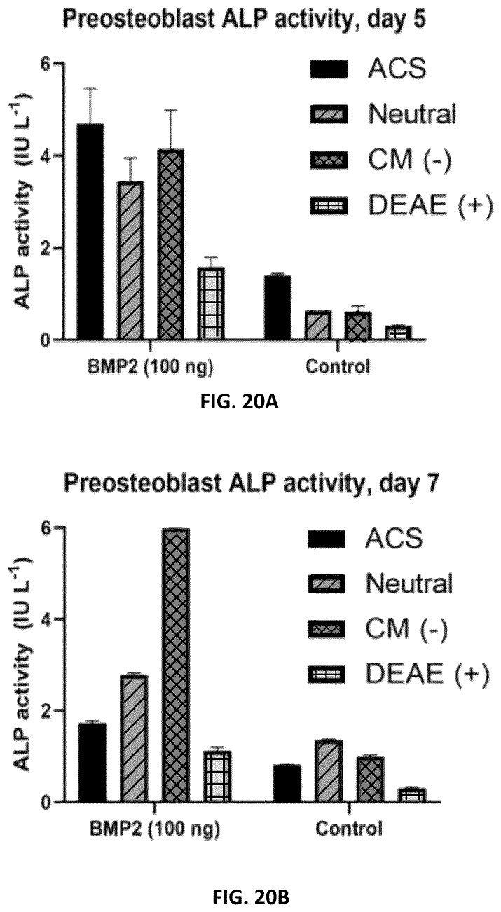

FIG. 20A and FIG. 20B are plots of ALP activity of human pre-osteoblasts cells seeded on collagen foams of varying charge formulations dosed with 100 ng of BMP-2 after 5 days in culture (FIG. 20A) and 7 days in culture (FIG. 20B). The testing has demonstrated increased ALP activity at 7 days with 100 ng of BMP-2 in negatively charged collagen foam (denoted as CM (−)) when compared to all other formulations. This value also increases when compared to the 5-day activity value. The data indicates that this is due to the negatively charged collagen foam holding on to the positively charged BMP-2 longer than the other charge formulations.

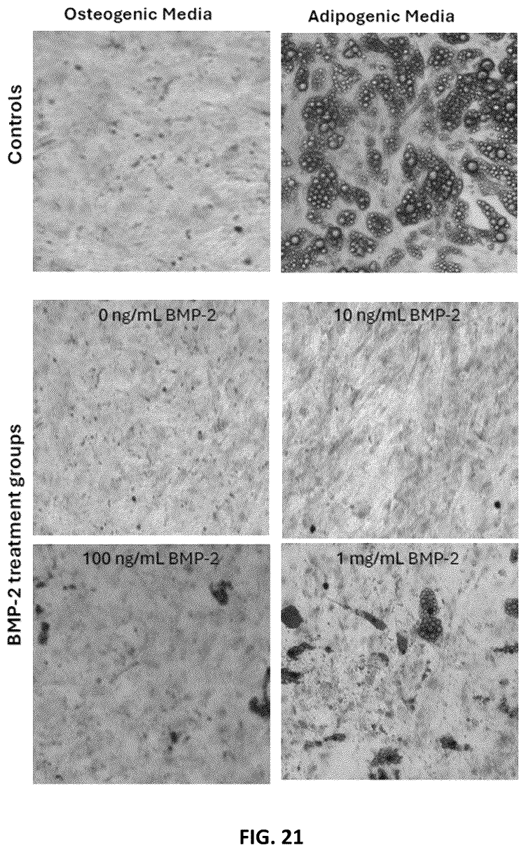

FIG. 21 illustrates off-target effects resulting from high BMP-2. Illustrated are control samples of osteogenic media and adipogenic media. Further illustrated are samples with four levels of BMP-2. The four levels are 0 ng/ml, 10 ng/ml, 100 ng/ml and 1 mg/ml. Adipogenic fat deposits produced by hMSCs are indicated in FIG. 21 with elevated Oil-Red O staining, likewise sensitive to high levels of rhBMP-2.

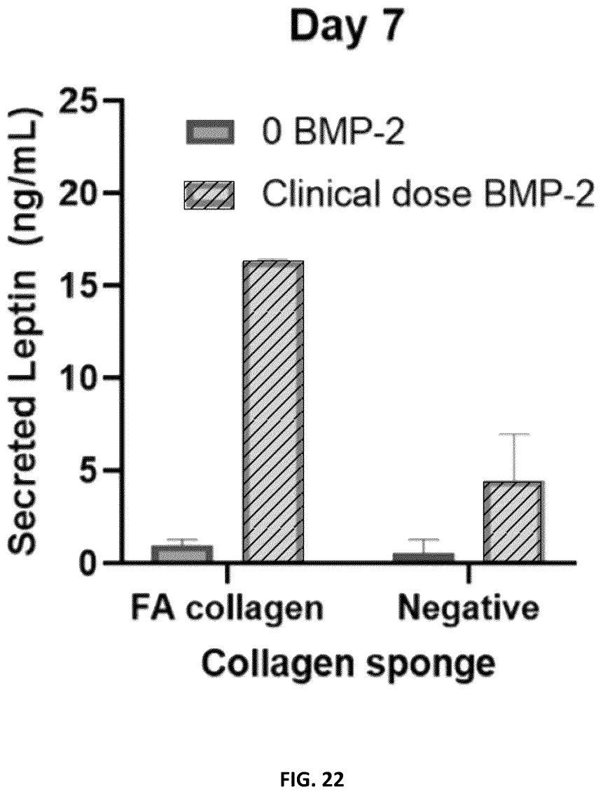

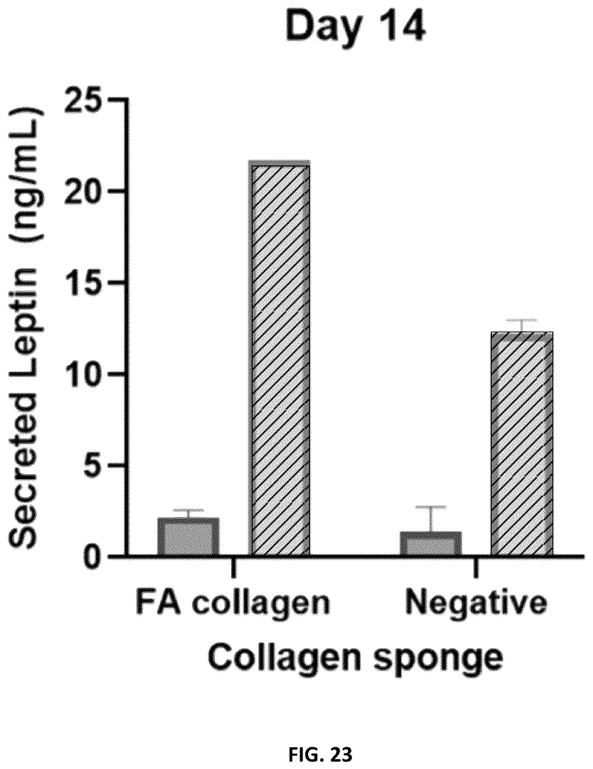

FIG. 22 and FIG. 23 are plots illustrating the effect of negatively charged collagen on lipid deposition. The graphs illustrate that negatively charged collagen mitigates the extent of the undesirable lipid deposition. In the graphs, an FA collagen sponge and a negatively charged collagen sponge are provided with a clinical dose of BMP-2. A level of secreted leptin is graphed for the two collagen samples and compared to samples without a clinical dose of BMP-2.

In FIG. 22, after 7 days, the FA collagen sample with a clinical dose of BMP-2 measured approximately 16 ng/ml of secreted leptin. The negatively charged collagen sample with a clinical dose of BMP-2 measured approximately 5 ng/ml of secreted leptin. The samples without the BMP-2 measured a negligible amount of secreted leptin.

FIG. 23 illustrates the same samples tested after 14 days. In this example, the FA collagen sample with a clinical dose of BMP-2 measured approximately 22 ng/ml of secreted leptin. The negatively charged collagen sample with a clinical dose of BMP-2 measured approximately 12 ng/ml of secreted leptin. The samples without the BMP-2 measured a less than 3 ng/ml of secreted leptin.

As illustrated, the negatively charged collagen serves to mitigate the extent of the undesirable lipid deposition.

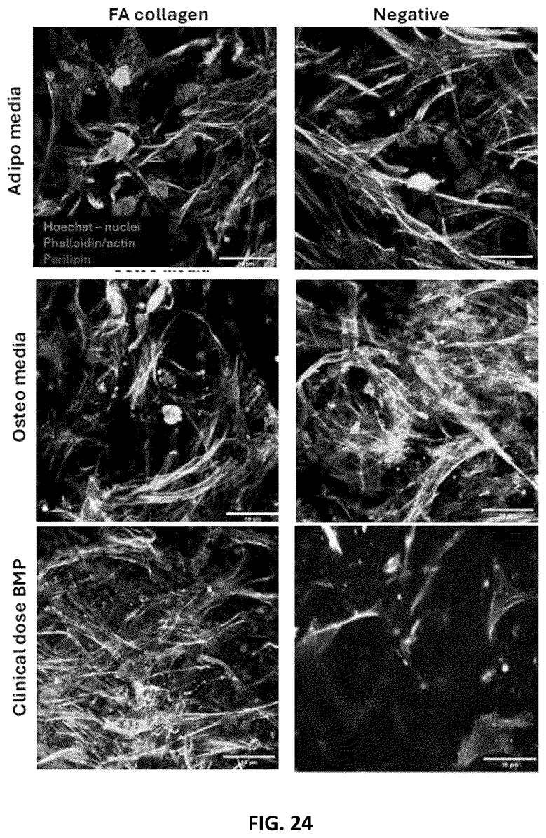

FIG. 24 is an example of perilipin staining to target maturing adipocytes. The mitigation of the undesirable lipid deposition is illustrated by comparing the FA collagen with the negatively charged collagen sample. The samples are illustrated with adipo media, osteo media, and a clinical dose of BMP-2. The maturing adipocytes are illustrated in red. The negatively charged collagen with the BMP-2 dose does not display any maturing adipocytes.

Based on the examples illustrated herein, the technology may be used to impact in vitro mineralization via the charged collagen platform by its interaction with rhBMP-2.

The examples illustrate that high levels of rhBMP-2 can elicit adipogenesis. The negatively charged collagen platform may mitigate lipid deposition while improving the osteogenic differentiation of hMSCs. Beyond the described use with BMP-2, data indicates that additional macromolecules of interest, such as EGF and PDGF-bb can be used to tune the charge of collagen with this platform to improve the binding affinity of macromolecules of interest.

### Example 19. Antimicrobial Effect of Positively Charged Collagen-Based Skin Substitute in Resisting Infection

Bacterial infection hinders healing following burn-related injuries and frequently leads to the development of chronic wounds. A pro-regenerative skin substitute with innate antimicrobial properties provides a potential solution for complex wound healing cases. Using a dextran-based crosslinker and a purified Type I collagen-based matrix, a collagen scaffold with innate antimicrobial resistance can be generated while maintaining key mechanical and regenerative properties. The charged collagen matrix, in particular positively charged collagen skin substitute, provides an ability to resist infection and support wound closure.

In vitro biocompatibility was assessed using human mesenchymal stromal cells and metabolic assays. Cells were seeded on type I collagen disks crosslinked with either formaldehyde (FA), electrically neutral dextran aldehyde (DA), positively charged diethyl aminoethyl-dextran aldehyde (DEAE-DA (+)), or negatively charged carboxymethyl-dextran aldehyde (CM-DA (−)). Metabolic activity was analyzed for intervals of 1, 3, and 7 days.

Wound closure efficacy and in vivo biocompatibility were determined using a porcine wound healing model. Twelve full thickness 3 cm×3 cm excisional wounds were made on each of the backs of three female Yorkshire pigs. Wounds were treated with one of bandaging only, glutaraldehyde cross-linked collagen/chondroitin-6-sulfate matrix (Collagen-C6S), CM-DA (−), DA, or DEAE-DA (+) matrices. Wounds were imaged at 3, 7, 11, and 14 days. Two pigs were sacrificed at 7-day and 14-day timepoints for histological analysis using Hematoxylin and Eosin (H&E) and Masson's Trichrome stains.

Antimicrobial efficacy was evaluated using AATCC Test Method 100 (modified). In the test, 1″×1″ samples of neutral DA and DEAE-DA (+) were inoculated with Streptococcus pyogenes 12344 (S. pyogenes), Staphylococcus epidermidis 35984 (S. epidermidis), Staphylococcus aureus USA 300 (MRSA), Escherichia coli 8739 (E. coli), Acinetobacter baumannii AB5075-UW (A. baumannii), or Klebsiella pneumoniae subsp. Pneumoniae 4352 (K. pneumoniae). Bacterial load was quantified at 0 and 24 hours.

In vitro, all groups had greater than 80% cell metabolic activity after 24 hours compared to time 0 except for the control FA-collagen. The test was continued for 7 days. The bacterial load was quantified at 3 and 7 days. The trends continued through to 3 and 7 days.

In vivo, no statistically significant differences in wound closure between the sample groups at any timepoint were identified. Tests conducted at 14 days indicated that cellular infiltration into the DEAE-DA (+) matrix was similar to the control Collagen-C6S, CM-DA (−), and DA groups. DEAE-DA (+) wounds also contained abundant new capillaries. Wounds treated with CM-DA (−) contained less mature neocollagen than those treated with Collagen-C6S. Neutrophils were still present in the CM-DA (−) wound at day 14 but in fewer numbers than at day 7.

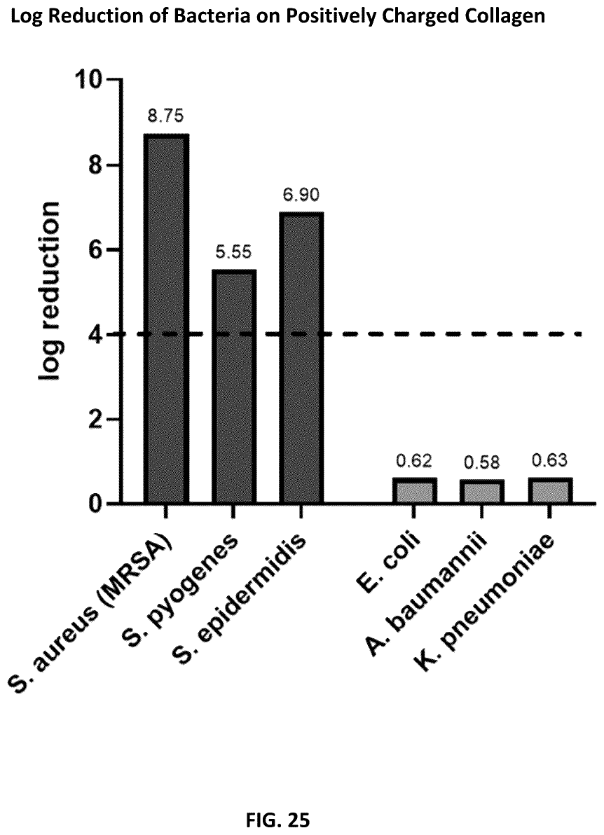

FIG. 25 is a graph illustrating log reduction results for bacteria on positively charged collagen.

In vitro, DEAE-DA (+) showed significant log reduction for all bacteria, compared to control DA (p<0.0001). Illustrated on the graph are the reductions for the bacteria after 24 hours of contact with DEAE-DA (+) compared to the negative control (DA). As illustrated, Gram-positive bacteria S. aureus showed a reduction of 8.75 log. Gram-positive bacteria S. pyogenes showed a reduction of 5.55 log. Gram-positive bacteria S. epidermidis showed a reduction of 6.90 log. The three Gram-negative bacteria, E. coli, A. Baumannii, and K. pneumoniae, showed less than 1 log reduction compared to the control.

The results illustrated on the graph indicate that DEAE-DA (+) is bactericidal for only Gram-positive bacteria based on a reduction that is greater than 4 log, as explained in AATCC TM 100-2019. DEAE-DA (+) collagen skin substitute performed similarly to the control Collagen-C6S in wound management applications. DEAE-DA (+) collagen showed antimicrobial activity towards Gram-positive bacteria, including MRSA.

In accordance with the disclosure herein, the following example embodiments may be provided or any combinations of the embodiments.

Embodiment 1 includes a method for preparing a cross-linked collagen matrix having a positive or negative charge, comprising providing a collagen dispersion; varying a charge density across the collagen dispersion by dissolving into the collagen dispersion one or more positively or negatively charged cross-linkers; and lyophilizing a mixture of the collagen dispersion and the one or more dissolved cross-linkers to create a collagen matrix.

Embodiment 2 includes the method of embodiment 1, comprising exposing the lyophilized collagen matrix to a physiologically or pharmaceutically active ingredient of an opposite charge to cause the collagen matrix to sequester at least a portion of the active ingredient.

Embodiment 3 includes the method of embodiment 1, wherein the cross-linker is selected from the group consisting of dextran aldehyde, DEAE-dextran aldehyde (positively charged), and CM-dextran aldehyde (negatively charged), or combinations thereof.

Embodiment 4 includes the method of embodiment 1, wherein a degradation profile of the collagen is controlled by a location and density of the one or more cross-linkers dissolved in the collagen dispersion.

Embodiment 5 includes the method of embodiment 4, wherein the degradation profile of the collagen matrix releases the active ingredient at a controlled rate and direction based on a type and location of the one or more cross-linkers.

Embodiment 6 includes the method of embodiment 2, wherein a charge of the collagen dispersion is negative, and the active ingredient is BMP-2.

Embodiment 7 includes the method of embodiment 1, wherein cross-linkers of different charges are arrayed across a single collagen matrix.

Embodiment 8 includes the method of embodiment 7, wherein an outer ring of the single collagen matrix has a particular charge and an inner portion inside the outer ring has a different charge than the particular charge.

Embodiment 9 includes a medical device comprising a collagen matrix, wherein the collagen matrix has a varied charge density across the collagen matrix created by a dissolved one or more positively or negatively charged cross-linker in a collagen dispersion, and then lyophilized; and a physiologically or pharmaceutically active ingredient of an opposite charge to the collagen dispersion that is sequestered in the collagen matrix.

Embodiment 10 includes the medical device of embodiment 9, wherein the cross-linker is selected from the group consisting of dextran aldehyde, DEAE-dextran aldehyde (positively charged), and CM-dextran aldehyde (negatively charged), or combinations thereof.

Embodiment 11 includes the medical device of embodiment 9, wherein the degradation profile of the collagen matrix is configured to release the active ingredient at a controlled rate and direction.

Embodiment 12 includes the medical device of embodiment 9, wherein a charge of the collagen matrix is negative, and the active ingredient is BMP-2.

Embodiment 13 includes the medical device of embodiment 12, wherein the collagen matrix is applicable in vitro to promote mineralization.

Embodiment 14 includes a method for treating a subject or patient in need of tissue regeneration, comprising providing a medical device comprising a collagen matrix, wherein the collagen matrix has a varied charge density across the collagen matrix created by a dissolved one or more negatively charged cross-linker and a physiologically or pharmaceutically active ingredient of an opposite charge to the cause the collagen matrix that is sequestered in the collagen matrix; and applying the medical device to the subject or patient.

Embodiment 15 includes the method of embodiment 14, wherein the active ingredient is BMP-2.

Embodiment 16 includes the method of embodiment 14, comprising applying the collagen matrix comprising sequestered BMP-2 in vitro to promote mineralization.

Embodiment 17 includes the method of embodiment 14, wherein the negatively charged collagen matrix mitigates lipid deposition while improving osteogenesis.

Embodiment 18 includes a method of controlling release of a physiologically or pharmaceutically active ingredient in a subject, comprising: administering to the subject a medical device comprising a cross-linked collagen matrix having a varied charge density across the collagen dispersion created by a dissolved one or more positively or negatively charged cross-linker, and a physiologically or pharmaceutically active ingredient of an opposite charge.

Embodiment 19 includes the method of embodiment 18, wherein the cross-linker is selected from the group consisting of dextran aldehyde, DEAE-dextran aldehyde (positively charged), and CM-dextran aldehyde (negatively charged), or combinations thereof.

Embodiment 20 includes a method for preparing a medical device, comprising providing a first collagen dispersion containing a first positively or negatively charged cross-linker to generate a first mixture; freezing the first mixture in a first mold to generate a first frozen mixture of a first shape; providing a second collagen dispersion containing a second positively or negatively charged cross-linker to generate a second mixture; pouring the second mixture adjacent to the first frozen mixture to generate a combination; and lyophilizing the combination to generate the medical device comprising: a first cross-linked collagen matrix portion having a positive or negative charge; and a second cross-linked collagen matrix portion having a positive or negative charge which is different from the charge of the first cross-linked collagen matrix portion in the type and/or level of charge.

Embodiment 21 includes a medical device comprising: a first cross-linked collagen matrix portion having a first charge, first charge density, and first cross-linking density; and a second cross-linked collagen matrix portion having a second charge, second charge density, and second cross-linking density, wherein the first cross-linked collagen matrix portion and the second cross-linked collagen matrix portion differ in the type of charge, charge density and/or cross-linking density.

Embodiment 22 includes a method for treating a patient in need of tissue regeneration, comprising: providing a medical device having a first cross-linked collagen matrix portion having a first charge, first charge density, and first cross-linking density; and a second cross-linked collagen matrix portion having a second charge, second charge density, and second cross-linking density, wherein the first cross-linked collagen matrix portion and the second cross-linked collagen matrix portion differ in the type of charge, charge density and/or cross-linking density; and applying the medical device to the patient.

Embodiment 23 includes a method for treating wounds in a patient, comprising: applying a positively charged cross-linked collagen scaffold to a wound, wherein the collagen scaffold facilitates wound healing while providing antimicrobial resistance.

Embodiment 24 includes the method of embodiment 23, wherein the positively charged cross-linked collagen scaffold is generated by applying diethyl aminoethyl-dextran aldehyde (DEAE-DA (+)) to a collagen scaffold.

Embodiment 25 includes the method of embodiment 23, wherein the positively charged cross-linked collagen scaffold has antimicrobial activity toward Gram-positive bacteria.

Embodiment 26 includes the method of embodiment 23, wherein the positively charged cross-linked collagen scaffold is generated by applying a positively charged cross-linker to a purified Type I collagen-based matrix.

Embodiment 27 includes a skin substitute that facilitates wound healing while providing antimicrobial resistance, comprising a collagen matrix with a positive charge created by a dissolved positively charged cross-linker.

Embodiment 28 includes the skin substitute of embodiment 27, wherein the positively charged cross-linker is positively charged diethyl aminoethyl-dextran aldehyde.

Embodiment 29 includes the skin substitute of embodiment 27, wherein the skin substitute has antimicrobial activity toward Gram-positive bacteria.

Embodiment 30 includes a method for preparing a medical device, comprising: providing a first collagen dispersion comprising a first cross-linker to generate a first mixture; freezing the first mixture in a first mold to generate a first frozen mixture of a first shape; providing a second collagen dispersion comprising a second charged cross-linker to generate a second mixture; pouring the second mixture adjacent to the first frozen mixture to generate a combination; lyophilizing the combination to generate the medical device comprising: a first cross-linked collagen matrix portion having a first charge, first charge density, and first cross-linking density; and a second cross-linked collagen matrix portion having a second charge, second charge density, and second cross-linking density, wherein the first cross-linked collagen matrix portion and the second cross-linked collagen matrix portion differ in the type of charge, charge density and/or cross-linking density.

The terms “collagen dispersion” and “collagen suspension” are used interchangeably here.

The terms “cross-linker”, “cross linker”, and “crosslinker” are used interchangeably herein. Likewise, the terms “cross-link”, “cross link”, and “crosslink” are used interchangeably herein.

As used in the specification and claims for the purposes of describing and defining the invention, the terms “about” and “substantially” represent the inherent degree of uncertainty attributed to any quantitative comparison, value, measurement, or other representation. The terms “about” and “substantially” also represent the degree by which a quantitative representation may vary from a stated reference without resulting in a change in the basic function of the subject matter at issue. “Comprise”, “include”, “have,” and variations of each word include the listed parts and can include additional parts not listed. “And/or” includes one or more of the listed parts and combinations of the listed parts.

One skilled in the art will realize the disclosure may embody other specific forms without departing from the spirit or essential characteristics thereof. The foregoing examples in all respects illustrate rather than limit the disclosure described herein. The appended claims, rather than the foregoing description, thus indicate the scope of the disclosure, and embrace all changes that come within the meaning and range of equivalency of the claims.

1. A method for preparing a cross-linked collagen matrix having a positive or negative charge, comprising:
providing a collagen dispersion;
varying a charge density across the collagen dispersion by dissolving into the collagen dispersion one or more positively or negatively charged cross-linkers; and
lyophilizing a mixture of the collagen dispersion and the one or more dissolved cross-linkers to create a collagen matrix.

2. The method of claim 1, comprising exposing the lyophilized collagen matrix to a physiologically or pharmaceutically active ingredient of an opposite charge to cause the collagen matrix to sequester at least a portion of the active ingredient.

3. The method of claim 1, wherein the cross-linker is selected from the group consisting of dextran aldehyde, DEAE-dextran aldehyde (positively charged), and CM-dextran aldehyde (negatively charged), or combinations thereof.

4. The method of claim 1, wherein a degradation profile of the collagen is controlled by a location and density of the one or more cross-linkers dissolved in the collagen dispersion.

5. The method of claim 4, wherein the degradation profile of the collagen matrix releases the active ingredient at a controlled rate and direction based on a type and location of the one or more cross-linkers.

6. The method of claim 2, wherein a charge of the collagen dispersion is negative, and the active ingredient is BMP-2.

7. The method of claim 1, wherein cross-linkers of different charges are arrayed across a single collagen matrix.

8. The method of claim 7, wherein an outer ring of the single collagen matrix has a particular charge and an inner portion inside the outer ring has a different charge than the particular charge.

9. A medical device comprising:
a collagen matrix, wherein the collagen matrix has a varied charge density across the collagen matrix created by a dissolved one or more positively or negatively charged cross-linker in a collagen dispersion, and then lyophilized; and
a physiologically or pharmaceutically active ingredient of an opposite charge to the collagen dispersion that is sequestered in the collagen matrix.

10. The medical device of claim 9, wherein the cross-linker is selected from the group consisting of dextran aldehyde, DEAE-dextran aldehyde (positively charged), and CM-dextran aldehyde (negatively charged), or combinations thereof.

11. The medical device of claim 9, wherein the degradation profile of the collagen matrix is configured to release the active ingredient at a controlled rate and direction.

12. The medical device of claim 9, wherein a charge of the collagen matrix is negative, and the active ingredient is BMP-2.

13. The medical device of claim 12, wherein the collagen matrix is applicable in vitro to promote mineralization.

14. A method for treating a subject or patient in need of tissue regeneration, comprising:
providing a medical device comprising a collagen matrix, wherein the collagen matrix has a varied charge density across the collagen matrix created by a dissolved one or more negatively charged cross-linker and a physiologically or pharmaceutically active ingredient of an opposite charge to the cause the collagen matrix that is sequestered in the collagen matrix; and
applying the medical device to the subject or patient.

15. The method of claim 14, wherein the active ingredient is BMP-2.

16. The method of claim 14, comprising applying the collagen matrix comprising sequestered BMP-2 in vitro to promote mineralization.

17. The method of claim 14, wherein the negatively charged collagen matrix mitigates lipid deposition while improving osteogenesis.

18.-30. (canceled)
